(19) United States
(12) Patent Application Publication
(10) Pub. No.: US 2012/0101165 A1
(43) Pub. Date: Apr. 26, 2012

(54) Urea Compositions With Cooling Effect

(76) Inventors: Fatemeh Mohammadi, Hauppauge, NY (US); Kathleen James-Lawrence, Valley Stream, NY (US); Barbara A. Crockett, Greenlawn, NY (US)

(21) Appl. No.: 13/090,615

(22) Filed: Apr. 20, 2011

Related U.S. Application Data
(63) Continuation-in-part of application No. 11/625,472, filed on Jan. 22, 2007, now abandoned.

(51) Int. Cl. A61K 8/44; A61Q 19/00

(52) U.S. Cl. 514/588

(57) ABSTRACT

The present invention includes a urea powder, the particles of which are protected in a dimethicone and silica mixture, prior to being incorporated into anhydrous compositions. The protected urea is capable of imparting a cooling activity to a topical composition. The invention also pertains to topical, cooling compositions containing a protected urea. The use of the protected urea to impart a cooling activity to topical anhydrous compositions is new. Topical, anhydrous compositions comprising the protected urea are also new. Anhydrous compositions containing the protected urea are stable against hydrolysis that might otherwise occur due to the ambient humidity. Disclosed compositions create a clean and fresh feel for a user.

This application is a continuation-in-part of U.S. Ser. No. 11/625,472, filed Jan. 22, 2007, and claims benefit thereof.

### FIELD OF THE INVENTION

The present invention pertains to topical compositions for skin care. Specifically, the principles of the present invention may be incorporated into various types of skin treatment compositions, to impart a cooling property to the compositions.

### BACKGROUND

Carbamide, also known as urea (systematic name: diaminomethanal), is a polar, crystalline solid up to about 132° C. Its molecular formula is:

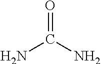

Urea has a high solubility in water: 108 g/100 mL at 20° C. and increasing up to 733 g/100 mL at 100° C. The reaction is endothermic, meaning that the urea-water system absorbs heat from its surroundings as the reaction proceeds. For urea, the heat of solution in water is about −57.8 cal/g. The use of urea and substituted ureas in topical compositions is known, having been used as an antiseptic, antibacterial, antifungal, antipruritic, a deodorizer and other uses. Urea possesses keratolytic activity (the removal of dead skin cells) and, at high concentrations, a protein solubilizing activity. It is identified as a skin penetration enhancer, both for itself and other actives targeted below the skin surface.

In the stratum corneum, water retention is primarily achieved by a high concentration of water-soluble hygroscopic molecules, collectively known as the natural moisturizing factor (NMF). The NMF binds water and acts as a reservoir to maintain tissue hydration. Urea is a humectant produced naturally within the stratum corneum as part of the NMF and plays a critical role in moisture retention in the skin. Furthermore, exogenous urea applied to the skin has high osmotic activity. Topically applied urea penetrates the skin by breaking hydrogen bonds in the outer layers of the stratum corneum to expose water-binding sites, below (a phenomenon known as hydrotopic solubilization). For this reason, urea may be used very effectively in topical moisturizing compositions. As a humectant, at concentrations lower than about 10%, a composition containing urea is intended as an everyday or occasional moisturizer. At concentrations significantly higher than about 10% (even up to 40%), topical compositions containing urea are effective treatments for severe cases of dry, rough skin, such as ichthyosis and psoriasis.

Despite their potent moisturizing properties, commercially viable cosmetic preparations containing urea are difficult to formulate. In aqueous systems, urea hydrolyzes steadily, such that, in less time than an ordinary product shelf-life, significant quantities of ammonia, other amines and carbon dioxide have formed in the product. Amines have a distinct, unpleasant odor and tend to raise the pH of the composition. For example, it has been reported (US 2004/0033963) that a 20% urea solution has an initial pH of about 7.4, and rises to 8.8, after 11 months at room temperature. Discoloration or other breakdown of the product, including phase separation, may also occur as a result of the hydrolysis of urea in an aqueous composition. This is completely unacceptable in personal care products.

Thus, it may seem that these problems are avoided when urea is incorporated into an anhydrous vehicle. However, urea exhibits a critical relative humidity. Critical relative humidity is the relative humidity of the surrounding environment at which a material begins to spontaneously absorb moisture from the air. Below the critical relative humidity the material does not absorb moisture from the air. The critical relative humidity of urea is about 81% at 20° C. and about 73% at 30° C. Therefore, in routinely encountered conditions, it is the tendency for urea to absorb ambient moisture, so that even if the urea-containing vehicle is anhydrous, unprotected urea is generally susceptible to ambient moisture. Therefore, the use of an anhydrous system does not completely avoid the problems noted above, because urea will hydrolyze from the presence of moisture in the air and the shelf-life of a anhydrous product containing urea may be shortened.

For these reasons, some products that seek the benefits of urea also incorporate measures (such as, buffering or the use of a urea derivative) to reduce the rate of hydrolysis of urea. For example: European Patent Application No. 0101887A2 discloses a cosmetic composition comprising an aqueous solution of a urea compound that is selected from the group consisting of urea, lower alkyl-substituted urea, and mixtures thereof, present at a concentration of about 0.5 to about 12 molar, and an amount of an ammonium salt of an unreactive acid sufficient to adjust the pH value of the urea compound solution to about 6 to about 8, the cosmetic composition being stable to the production of titratable alkali. Preferred ammonium salts, according to this patent application, include ammonium salts of strong acids such as carboxylic acids having up to four carbon atoms.

JP 59020217 describes an aqueous, jelly-like composition containing between 1 and 48% by weight of urea, an ammonium compound and a carboxyvinyl polymer. The pH of the composition is adjusted to 5.5 to 7.5, by adding a base made up of hydroxides of alkali metals, alkanolamines, basic amino acids and aqueous ammonia. The reference teaches that the combination of all the components present in the disclosed composition synergistically provides for inhibition of the decomposition of urea. It is therefore implied in this patent that the stability of the composition would be reduced if any of the constitutional components would be missing.

U.S. Pat. No. 6,355,259 discloses aqueous cosmetic compositions comprising urea, allantoin and a urea-stabilizing buffer, comprising disodium hydrogenphosphate and citric acid or comprising potassium dihydrogenphosphate and disodium hydrogenphosphate, or both. Notwithstanding the inclusion of urea, which is useful for preventing rough dry skin and maintaining moisturization, the cosmetic composition, reportedly, does not have problems such as an increase in pH and an odor from ammonia produced by urea during storage for a long period of time.

As is clear, the prior art is replete with urea-containing compositions that use buffering to steady the pH and stabilize the urea, to the extent possible. However, buffered systems create electrolytes which may cause difficulties when working with traditional thickening systems. A brief survey of references that disclose urea containing topical compositions includes the following.

U.S. Pat. No. 3,507,797 discloses toilet bars made by forming an aqueous solution of 35-60% urea; 20-50% alkali metal, ammonium or substituted ammonium soap; and 7-15% of saturated C10-C20 aliphatic monocarboxylic acid; and heating to 95° C.-110° C., until there is just 2-4% water remaining. The toilet bar does not utilize a mixture of urea, silica and dimethicone as disclosed herein.

Preparation and characterization of biodegradable urea-loaded microparticles as an approach for transdermal delivery (Journal of Microencapsulation, September 2006; 23(6): 698-712) discloses urea encapsulated in microparticles on the order of less than 10 μm in diameter. The encapsulating material may be ethyl cellulose (EC), cellulose acetate phthalate (CAP) or poly(D,L-lacto-co-glycolic acid) (PLGA). The purpose of the encapsulation is to protect the urea from hydrolysis degradation before it can be delivered beneath the skin, and to specifically protect the urea from hydrolyzing on the skin surface (page 699, lines 29-31). Two of the encapsulating materials (EC, CAP) were considered ineffective for the intended purpose. The PLGA microparticles were on the order of 0.6-5.1 μm, which the article specifically says is sufficiently small to enter beneath the skin (see page 710). The reference does not report any cooling effect, nor mixing with dimethicone and silica.

U.S. Pat. No. 3,666,863 discloses a skin-treating composition comprising an aqueous solution of urea and lactic acid, the urea being present in an amount of from about 2 to 30 weight percent, the lactic acid being present in an amount of from about 0.5 to 8 weight percent, each range being based on the weight of solution. The reported advantage of this skin-treating composition is a decrease in the decomposition of the urea to form of carbon dioxide and ammonia, due to the lactic acid.

U.S. Pat. No. 4,137,302 discloses an anhydrous composition that prolongs the relatively short-lived softening and moisturizing benefits of urea by employing, instead of urea, a special derivative thereof, which does not breakdown with the release of ammonia. The reference discloses a composition for skin treatment comprising from 1 to 99% by weight of monoacetyl urea, together with a cosmetically acceptable vehicle other than water. Thus, even though a substituted urea was used, the reference teaches that an anhydrous system is required.

U.S. Pat. No. 4,518,614 discloses aqueous moisturizing compositions containing gibberellic acid and lysine, and, optionally, urea up to 5%. U.S. Pat. No. 4,694,021 discloses a method of treating tissue scars, which includes applying an aqueous composition containing urea, an argillaceous clay material, a p-hydroxybenzoic acid ester (i.e. methyl- or propylparaben), and a sulfosuccinate compound.

U.S. Pat. No. 5,100,674 discloses a process for preparing an effervescent aqueous solution having an emollient action on the cuticle of the nails, wherein solid, anhydrous compounds are simultaneously added into an aqueous medium, the compounds being urea, at least one alkali metal and/or ammonium bicarbonate and pyrrolidonecarboxylic acid (PCA), and at least one water-soluble ingredient having an emollient or pharmaceutical action for the treatment of the nails; said process further including introducing at least one neutral or basis amino acid. On being introduced into water, urea and the alkali metal or ammonium bicarbonate dissolve. Pyrrolidonecarboxylic acid reacts gradually with the alkali metal and/or ammonium bicarbonate to form a salt of pyrrolidonecarboxylic acid, which is soluble in water, which gives off carbon dioxide and which is hydrating agent, the action of which complements the emollient action of the urea. A solution is thereby obtained which does not attack the nails and the skin of the fingers in proximity to the nail, while having an efficacious emollient action for removing the cuticles. In addition, as a result of the evolution of carbon dioxide, the solution is effervescent, which adds to the comfort on using it.

U.S. Pat. No. 5,993,857 discloses an aqueous, non-oily suspension of microspheres of a polyoxymethylene urea. The urea microspheres are filled with skin moisturizing agents and provide a mild abrasive action when rubbed on the skin before rupturing to release their contents.

U.S. Pat. No. 6,010,707, U.S. Pat. No. 6,093,412 and U.S. Pat. No. 6,335,024 disclose moisturizing agents of aminoalcohol derivatives containing a urea functional group.

U.S. Pat. No. 6,284,281 discloses mattifying cosmetic compositions comprising particles of urea-formaldehyde resin and the use of such compositions to soften skin relief defects.

U.S. Pat. No. 6,660,251 discloses aqueous compositions of biotin and urea or urea derivative, wherein the proportion of the urea or urea derivative is from 1 to 40% by weight based on the total composition. The urea is said to bring about increased penetration of biotin into the skin. The penetration increase is dependent on the amount of urea used.

US 2003/0031688 discloses aqueous skin moisturizing compositions comprising a synergistic combination of coenzyme Q₁₀ and urea. Dimethicone is optional in the composition.

US 2004/0220137 discloses the use of one or more osmolytes and, optionally, one or more electrolytes, polyols or urea, in preparations to be applied topically for the treatment and active prevention of dry skin.

US 2004/0228894 discloses moisturizing aqueous compositions comprising urea and a sesquiterpene alcohol, and optionally, dimethicone.

US 2004/0033963 discloses compositions that include a molecular complex between urea and a functional substance that has at least one hydroxyl group and one carboxyl group, either as a free acid, a salt, an amide or a lactone. Reportedly, the compositions are stable when compared to conventional urea-containing compositions, and provide controlled-release of the urea into the skin.

US 2005/0037040 discloses topical aqueous compositions containing urea and alpha-hydroxy acid and/or an ammonium salt thereof, such as ammonium lactate.

US 2005/0042182 discloses foamable aqueous compositions of urea and a propellant in a cosmetically acceptable carrier.

US 2005/0112153 discloses dermatological aqueous compositions of urea and at least one emollient.

In US 2005/0181999, urea is a “pro-penetrating” agent for a biologically active agent, such as an anti-fungal agent. These compositions are substantially non-aqueous (less than 5% of the solvent system).

US 2006/0083708 discloses aqueous compositions of urea, mineral salts and tea tree extract for anti-itch and exfoliating treatments. Dimethicone may also be included in the composition as an emollient.

US 2006/0216260 discloses skin peeling compositions comprising at least 20% by weight of urea in an acceptable aqueous medium that is suitable to leave on the skin for at least five minutes and up to six hours, and that can be rinsed from the skin with water.

Topical cooling compositions comprising imidazolidinyl urea or diazolidinyl urea are known. Imidazolidinyl urea (C₁₁H₁₆N₈O₈) and diazolidinyl urea (C₈H₁₄N₄O₇) are decidedly different from urea (a.k.a. carbamide, a.k.a. diaminomethanal—CH₄N₂O) and the similarity in the names of these compounds suggests little about their respective behaviors in arbitrary topical compositions. Furthermore, there are commercial products that purport to provide a cool and refreshing feeling and that contain silica and dimethicone with either imidazolidinyl urea or diazolidinyl urea. One example of the former is marketed under the name Physician's Formula AquaPowder and of the later, John Abate Accelerating Cooling Bronzer Tingle Free. In neither of these products, nor in similar products, is the imidazolidinyl urea or diazolidinyl urea premixed with silica and dimethicone in a way that protects the urea as herein described. The imidazolidinyl urea or diazolidinyl urea are known preservatives and, to the best of the applicant's knowledge, do not provide a cooling effect. Furthermore, it would appear that any reported cooling effect achieved by these products is provided by a release of water from an entrapment in silica and dimethicone. Thus, the cooling effect is comparatively mild and based solely on a sudden contact of water to the skin. This is different from the comparatively dramatic cooling effect of the present invention, which is based on an endothermic reaction of urea in the immediate presence of the skin.

U.S. Pat. No. 5,356,627 discloses the use of a dimethicone-silica copolymer (Dow Corning® 7-4116) as a waterproofing agent, in mascara. The dimethicone and silica are not specifically used as a surface treatment. Urea is not disclosed.

None of the foregoing discloses particles of urea protected by being premixed in dimethicone and silica, nor the use of such to impart a cooling property to a topical composition. The applicants know of no compositions containing such a urea, nor the use of such compositions to provide a cooling sensation to the skin.

### OBJECTS OF THE INVENTION

A main object of the present invention is to provide a stable, topical composition comprising urea, that imparts to the skin a cooling sensation that is soothing and beneficial to the skin.

Another object of the invention is to provide a method of imparting to the skin a cooling sensation that is soothing and beneficial to the skin.

Another object of the invention is to provide a method of imparting a cooling property to a topical composition.

### SUMMARY OF THE INVENTION

These and other objects of the invention are met by the use of a urea powder, the particles of that are premixed with dimethicone and a specialized silica, prior to being incorporated into anhydrous compositions. The use of this premixed urea to impart a cooling activity to topical anhydrous compositions is new. Topical, anhydrous compositions comprising the premixed urea are also new. The premixed urea is protected, at least partially, against hydrolysis that might otherwise occur due to ambient humidity. Nevertheless, when a composition according to the present invention is applied to wet skin or applied to dry skin and subsequently moistened, the urea reacts endothermically with the water. The absorption of heat by the topically applied premixed urea provides a significant cooling sensation to the skin. The urea rinses easily and leaves no residue. To provide the cooling sensation, the urea needs to be on the skin for only a short time (i.e. several seconds or less than one minute). Some of the urea applied to the skin may provide a moisturizing and penetrating effect, which, combined with the cooling sensation, creates a clean, fresh feel. The cooling effect may also be used as a signal to a user that the product is efficacious. The degree of cooling can be regulated by the concentration of premixed urea in the composition. Compositions of the invention may include any other ingredients that are suitable for anhydrous, topical preparations. The use of the premixed urea places no restriction on the manufacturing process (i.e. not heat sensitive). All types of treatment products may be enhanced by the inclusion of a premixed urea according to the present invention. Generally, no other special ingredients are needed (i.e. no special preservatives for the urea). Generally, compositions according to the present invention may be packaged in any conventional packaging; for example, a jar, a tube, an impregnated wipe, a dual compartment container wherein one compartment holds the anhydrous, premixed urea composition and the other holds an aqueous phase; etc.

### DETAILED DESCRIPTION

### Terminology

Throughout this specification, the terms “comprise,” “comprises,” “comprising” and the like shall consistently mean that a collection of objects is not limited to those objects specifically recited.

“Substantially anhydrous” means that it may be possible to formulate efficacious compositions according to the present invention by including compounds that comprise incidental amounts of water.

The term “protected” in relation to urea means that urea has been mixed with silica and dimethicone in such a way that the combination of dimethicone and silica inhibits at least some of the urea from reacting with water, either in the composition or in the ambient atmosphere.

“Cooling”, “cooling sensation” or other semantic equivalents mean that a topical application of a composition according to the present invention causes the temperature of the skin to drop by an amount that can be perceived by a user.

“Effective amount” and “cooling-effective amount” mean an amount that is sufficient to provide a “cooling sensation”.

“Activate” means to initiate an endothermic urea hydrolysis reaction, which reaction proceeds at a rate such that a cooling sensation is achieved.

### Features of a Protected Urea

As noted, the use of an anhydrous or substantially anhydrous vehicle is generally insufficient to avoid an unacceptable degradation of a urea-containing product. Thus, the present invention includes means for protecting the product from the ambient humidity and from any incidental water that may be present in the composition. Thus, significant product degradation is avoided and the functionality of the urea is maintained until the time of use.

Advantageously, at the time of use, a topically applied composition according to the present invention is capable of reacting with additional water (from a tap, for example) in the characteristic endothermic solubility reaction, thus producing a powerful cooling effect. In other words, the protected urea described herein, does not substantially react with ambient moisture, nor with incidental water (see below) that may be present in the composition, but it does substantially react with water that a user typically applies in routine washing or bathing. Thus, in this description, it is important to draw a distinction between ambient moisture to which a composition of the present invention may be exposed, and water that is deliberately applied by a user to activate an endothermic solubility reaction. The principle difference between these is the amount of water relative to the surface area of protected urea and the degree of friction applied to the protected urea. Ambient moisture is unable to activate the protected urea, as we have defined “activate”, above.

Such compositions are new, unexpected and non-obvious over the prior art. The protection imparted to the urea is capable of preventing substantial degradation of the product during storage and during the shelf-life of a product containing the raw material. However, when applied to the skin and contacted with substantial quantities of water, the urea treatment does not prevent an endothermic reaction of the water and urea. The features just described may be met by a mixture of urea powder silica and dimethicone.

Although silica-dimethicone surface treatments of some materials are known, it was hitherto unknown to provide a mixture of urea, silica and dimethicone for deployment in a cooling composition. One example of a prior use of silica and dimethicone as a surface treatment, is in a product called Natural Hot™ TR, by Distinctive Cosmetics Ingredients, LLS (Plainfield, N.J.). In that product, calcium chloride is surface-treated or encapsulated with silica and dimethicone. Calcium chloride reacts exothermically with water to give off heat. The main benefit of the surface treatment is realized during use of the product, to control the rate of the exothermic reaction and extend the time over which heat is released. This is unlike the present invention where the main benefit of the silica and dimethicone is realized prior to the use of the product, to stabilize the urea against degradation caused by ambient humidity. Also, the treated calcium chloride is exothermic in water, while the protected urea is endothermic. Thus, the premixed urea of the present invention is different from the treated calcium chloride of the prior art, and used differently to produce a different effect.

### Other Requirements of the Composition

Within an effective composition according to the present invention, the integrity of the protected urea must be maintained until the time of use. The most straightforward means for accomplishing this is to provide the urea in a cosmetically or dermatologically acceptable, anhydrous vehicle, or at least in a substantially anhydrous vehicle. Based on the specifics of the composition, the total amount of water that may be allowed in efficacious compositions of the present invention will vary, but may readily be determined by routine experimentation. Roughly, we expect that substantially anhydrous efficacious compositions may be possible when the total water content is below about 5% by weight of the composition. Preferably, the composition contains no more than about 5% by weight of water, more preferably, no more than about 1%, and, most preferably, no more than about 0.1% by weight of water.

Alternatively, as long the protected urea in the composition is inert with respect to any water that may be present in the composition, then the spirit and functionality of the invention are maintained. Therefore, compositions that, by whatever means, are able to preserve the integrity of the protected urea, are within the scope of this invention. For example, a composition comprising a protected urea and water that is encapsulated or otherwise segregated from the protected urea, could, in principle, be an effective cooling composition.

### Effectiveness of the Composition

The degree of cooling effect brought about specifically by the protected urea component depends on several factors, including the concentration of the protected urea in the composition at the time of use. Useful compositions according to the present invention may comprise, by weight, from about 0.1% to about 50% of protected urea. Preferably, useful compositions will comprise from about 1.0% to about 30% of protected urea and most preferably, from about 2% to about 20% of protected urea. The protected urea comprises 50%-90% urea, 5%-25% dimethicone, 5%-25% silica. An effective protected urea pre-mixture is available from Distinctive Cosmetics Ingredients, LLS (Plainfield, N.J.), as Natural Cool U-TR.

Table 1 demonstrates the effectiveness of the protected urea of the present invention at providing a cooling sensation to the skin. Natural Cool U-TR (a premix of urea (89%), dimethicone (6%) and hydrated silica (5%)) was made into a 50% aqueous solution. The temperature of the water solvent was measured over several minutes. As table 1 shows, for about the first to two minutes, the temperature of the solvent drops dramatically, and then begins to rise.

TABLE 1

| elapsed time (minutes) | temperature (° C.) |
|---|---|
| 0 | 23.3 |
| 0.5 | 22.2 |
| 1 | 8.8 |
| 2 | 9.3 |
| 3 | 10.2 |
| 4 | 11.0 |
| 5 | 11.8 |

Generally, the degree of cooling will also depend on factors in the control of the user, such as: the amount of the composition applied to the skin, the area of the skin to which the composition is applied and the time that the activated composition is left on the skin. There are virtually limitless combinations of these factors that may be useful and an individual user will, by routine trial end error, easily learn to use a composition of the present invention to good effect. However, by way of example, compositions of the present invention are effective when at least 1 gram of the composition is applied to a portion of the face (cheeks, for example), and then the face is rinsed by cupping water in the hands and splashing it against the face.

Some embodiments of the invention include a topical multi-phase composition comprising a pre-mixture phase (or first phase) and a substantially anhydrous phase (or second phase). The first phase is a mixture of 50%-90% urea, 5%-25% dimethicone, and 5%-25% silica. This phase is premixed before adding to the second phase. The second phase comprises a cosmetically acceptable vehicle. The first phase and the second phase are mixed in a ratio from about 1:1 to about 1:99; such that the total water content of the final composition is less than about 5%.

### Adjuvants

Other than the total water content, there are, in principle, no restrictions on the types of components in a composition according to the present invention. Compositions of the present invention may contain all manner of cosmetic, sensorial, dermatologic or nutraceutical ingredients. Preferably, when included, such adjuvants are present in the composition in amounts that do not destabilize the formula. A wide range of topically acceptable materials may advantageously be used to preserve or alter the physical properties of the composition in order to create for the user a unique and pleasurable sensorial experience and/or to provide a benefit to the skin. These materials include, but are not limited to, an effective amount of one or more of the following agents: abrasives, absorbents, anti-acne, anti-ageing, anti-wrinkle, antifungal, anti-inflammatory, antimicrobial, antioxidant, antiperspirant, antidandruff, anti-dermatitis, antipruritic, anti-emetic, anti-hyperkeratolytic, anti-dry skin, anti-psoriatic, anti-seborrhea, anti-asthmatic, astringents, bronchodilators, biocides, chemical exfoliants, cleansers, colorants, corticosteroids, deodorants, depigmenting, depilating, emollients, epilating, analgesics, hair conditioners, hormones, humectants, light-interacting, luster-imparting, make-up removers, pH adjusters, powders, rheological modifiers, shine-imparting, skin bleaching, skin conditioning, skin protecting, tanning, UV screening, vitamins and wound-healing. Just about any cosmetic, dermatologic or pharmaceutic agent suitable for topical use is within the purview of this invention, the only requirement being that an adequate amount of the protected urea remain stable prior to use.

### Forms of the Composition

Other than those restrictions imposed by the absence of water, there are, in principle, no restrictions on the form of a composition or vehicle comprising a composition according to the present invention. Generally, any conventional form of finished cosmetic product is possible. For example, cooling compositions comprising protected urea may be implemented as a cream, a lotion, an emulsion, a gel, a wax, a paste, a stick, a foam, liquid, etc. These product forms may be used for a number of applications, including but not limited to, hand and body lotions, cold creams, facial moisturizers, anti-acne preparations, topical analgesics, soaps or color cosmetics, including foundations, eyeshadows, lipsticks and the like.

### Packaging

In general, compositions according to the present invention may be scooped, poured, sprayed, pumped, squeezed, wiped, etc., from an appropriate container. Furthermore, even though the urea is generally protected in the silica-dimethicone mixture, not all of the urea may be perfectly protected. Therefore, to maximize the efficacy of a composition according to the present invention, the composition, when not in use, is preferably protected from ambient moisture. For example, a composition may be stored in a sealed container that inhibits water transmission. Plastic, glass and metal containers with sealing closures generally meet this criterion. If a composition according to the present invention is to be distributed in a flexible packaging (i.e. tube or packette), then the flexible packaging should inhibit substantially all water transmission through the flexible packaging. Because lotion pumps typically leave some product in the exit nozzle, lotion pumps that have self-sealing orifices may be preferred.

As noted above, the degree of cooling that a user receives depends on a number of factors that are in the control of the user. For this reason, it may be useful to provide the user with instructions for use of a product according to the present invention. This may typically be done by providing printed directions to the user, for example, in connection with the purchasing of the product. A main purpose of such directions is to maximize the benefit delivered to the user. Therefore, such directions may suggest one or more of the following: how much product to apply to a given part of the body, how to activate the endothermic urea reaction, how long to leave the product on the skin, how often to repeat the treatment, how to maintain a treatment regimen, what should be the follow up treatment, etc.

### Various Advantages of the Invention

An advantageous feature of the present invention is that the use of a protected urea does not impose any special conditions on the manufacture of compositions according to the present invention. No special equipment or special or extreme processing conditions are required. For example, the protected urea imposes no restriction on the temperature of the batch.

Another advantageous feature of the present invention is that any existing, anhydrous, cosmetically acceptable product may be improved by incorporating a cooling effect into that product. This may be done, for example, by providing a first composition which is cosmetically acceptable and anhydrous and adding to the first composition a protected urea component. Preferably, the protected urea component is evenly distributed throughout the first composition, as by simple mixing, for example.

Other advantageous features include: the urea leaves no residue on the skin because it is easily rinseable; the urea will provide some moisturization of the skin; that the composition is both cooling and moisturizing means a clean fresh feel is achieved; after use, the skin is better prepared to receive other actives or treatments due to the relaxation and opening of the pores of the skin and due to the skin penetrating ability of urea; the cooling sensation serves as a signal to let user know that the product is efficacious.

### How to Use

A preferred method of use (or treatment method) comprises spreading an effective amount of a composition comprising a protected urea, evenly over an area of the skin, contacting the urea with a sufficient quantity of water to activate enough of the urea to produce a desired temperature change in the skin; allowing the urea to remain on the skin for at least about five seconds: removing the composition from the skin. A method of use may further include, massaging the activated composition into the skin; repeating the treatment; following the treatment with a secondary dermatological treatment chosen to have some cumulative or synergistic effect with the urea cooling treatment. Preferably, the urea cooling treatment is performed at least once per week, more preferably at least once per day and most preferably at least twice per day, when the fullest benefit may be realized.

What we claim is:

1. A topical multi-phase composition comprising:
a pre-mixture phase of 50%-90% urea, 5%-25% dimethicone, and 5%-25% silica;
a second phase comprising a cosmetically acceptable vehicle;
wherein the ratio of first phase to second phase is from about 1:1 to about 1:99; and
where the total water content of the composition is no more than 5%.

2. A composition according to claim 1 which when applied to the skin and activated, is capable of causing a skin surface temperature to drop by at least 5° C.

3. A composition according to claim 1, in combination with instructions for use, wherein the instructions suggest one or more of the following: how much of the composition to apply to a given part of the body, how to activate the composition, how long to leave the composition on the skin, how often to repeat the application, how to maintain a treatment regimen, and what should be the follow up treatment.

4. A method of treating the skin of user comprising:
providing a composition according to claim 1;
spreading an at least 1 gram of the composition over an area of the skin;
contacting the composition with a sufficient quantity of water to activate the urea;
allowing the composition to remain on the skin for at least about five seconds;
removing the composition from the skin.

5. The method of claim 4 further comprising one or more steps of:
massaging the activated composition into the skin;
repeating the treatment;
following the treatment with a secondary dermatological treatment chosen to have some cumulative or synergistic effect with the urea cooling treatment.

6. A skin treatment regimen comprising the method of claim 4, wherein the method is performed at least once per week, more preferably at least once per day and most preferably at least twice per day.
